(19) United States
(12) Patent Application Publication
(10) Pub. No.: US 2004/0176783 A1
(43) Pub. Date: Sep. 9, 2004

(54) Vascular suturing clip

(75) Inventors: John K. Edoga, Morristown, NJ (US); Thierry Richard, Florham Park, NJ (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Edrich Vascular Devices, Inc., Morristown, NJ

(21) Appl. No.: 10/737,463

(22) Filed: Dec. 16, 2003

Related U.S. Application Data
(60) Provisional application No. 60/433,691, filed on Dec. 16, 2002.

(51) Int. Cl. A61B 17/10; A61B 17/06

(52) U.S. Cl. 606/139; 606/222

(57) ABSTRACT

A clip for suturing opposed ends of severed tissue. The clip has a body with a first end terminating in a needle and a second end having an opening thereat. The body may also include a bite portion between the first and second ends applying a biasing force to hold the suturing clip in either a default open or default closed condition, wherein the needle is remote from the opening in the open condition and in proximity to the opening in the closed condition. The body may also include a footpad adjacent the needle, wherein the footpad pinches the opposing ends of the severed tissue against the body adjacent to the opening when the clip is in the closed condition. The clips may be applied by a surgical device, which is also disclosed.

### CROSS REFERENCE TO RELATED APPLICATIONS

The present invention relates to U.S. Provisional Patent Application Serial No. 60/433,691 filed Dec. 16, 2002, the disclosure of which is hereby incorporated herein by reference.

### BACKGROUND OF THE INVENTION

The present invention relates to surgical clips, methods of using surgical clips, and devices for installing surgical clips. Surgical clips, such as those for closing external wounds or for anastomosis of internal vessels, are well known in the art. Methods of using such clips are also well known. Often, such clips are referred to as suturing clips.

The suturing clips of the prior art are typically formed from metal and may be bent into position by the surgeon or other medical professional. Many such clips are held in place by merely by pressure, as the they pinch the wounded tissue. Such clips require skill and judgment to determine the amount of closing pressure required for a particular injury. As such, there is a risk that the clip will be installed with too great a pressure, or too little pressure to properly heal the wound.

In addition, many of the prior art clips include teeth that are utilized to “bite” into the wounded flesh or vessel. These teeth are typically intended to assist with holding of the wounded flesh or vessel in place. Nevertheless, the teeth may often interject additional complications, such as causing localized tearing around the teeth. If such tearing is left unaddressed, it may spread to the edge of the wound such that the skin or vessel will be completely ruptured and no longer be secured by the teeth, rendering the clip non-functional. In addition, the localized tearing may become infected, even if limited to only a small amount.

Staples are also known for the closing of wounds. Staples are preferred in many instances. However, staples pierce the wounded tissue, often leading to additional complications.

Other prior art devices do not employ piercing teeth. Rather, such devices typically rely on pressure generated on a bearing surface through springs or through the resiliency of the clip itself to secure the wound. Such clips may not be capable of generating sufficient force to hold the wound closed without interjecting additional complications. Also, such clips are often not installed with sufficient pressure to permit them to be secured to the wound for the requisite period of time required for the wound to heal. Even if the clips are installed with a proper pressure, necrosis of the tissue may cause the tissue to thin, permitting the clip to slide off. This is especially troublesome for clips that do not include a biasing pressure, but merely retain a predetermined position upon setting.

When installed externally, such defects are readily curable. However, when installed internally, for example in carotid batch fixation, additional complications arise, in that the clips may only be fixed through surgical intervention.

Accordingly, it would be beneficial to provide a suturing clip having a combination of a piercing member and a bearing surface, such that the benefits of each of the prior art styles is employed without the inherent limitations of each. It would also be beneficial to provide clips having additional features, even if such features did not include the combination of a piercing member and a bearing surface.

### SUMMARY OF THE INVENTION

The aforementioned shortcomings of prior art suturing clips, and others not specifically listed, have been addressed by the present invention, which in one embodiment comprises a suturing clip comprising a body having first and second ends. The first end may terminate in a needle and the second end may form an opening thereat. The body may be adapted to manipulate the needle between a first and second position, the needle arranged spaced from the opening when in the first position and at least partially within the opening when in the second position. The first end may further comprise a hemostatic footpad where the footpad has a dimension larger than the opening such that the footpad may not pass through the opening when the clip is in the closed position. The body may further comprise a spring between said first end and said second end, the spring applying a biasing force to hold the body in either the first position or the second position. The needle may be deformed when in the second position such that the deformed needle interferes with the opening to prevent the body from being placed in the first position.

In other embodiments, the body may further comprise a bite portion between the first end and said second and a first needle arm extending from the bite portion to the first end. First and second extension members may extend from the bite portion to the second end. The first needle arm, the first extension member and the second extension member may be connected to each other by a pin at the bite portion. The first extension member and the second extension member may be placed in proximity to each other at the second end to form the opening.

In further embodiments, the suturing clip may comprise a body having a first end, a second end and a bite portion therebetween. The first end may terminate in a needle and the second end may terminate with a needle receiving ring adapted to receive the needle, wherein the bite portion applies a biasing force to hold the suturing clip in either one of a default open or a default closed condition, the needle being remote to the needle receiving ring in the open condition and in proximity with the needle receiving ring in the closed condition.

In further embodiments, the suturing clip may comprise a wire strand having a first end tapering into a needle, a second end configured to form a needle receiving ring to receive the needle, and a bite portion therebetween. The bite portion may bias the clip in either one of an open condition in which the needle is remote from the needle receiving ring and a closed condition in which the needle is at least partially within the needle receiving ring.

In a still further embodiment, the suturing clip may comprise a first extension arm, a second extension arm and a needle arm, the first extension arm and the second extension arm being pivotable about a bite portion of the clip to form a needle receiving ring, the needle arm terminating with a needle opposite the bite portion, wherein the needle may penetrate the needle receiving ring to place the clip in a closed condition.

In still further embodiments, a method of suturing opposing ends of severed tissue with a clamp having a body a first end terminating in a needle and a second end forming an opening thereat is disclosed. The method may comprise placing the opposing ends of the tissue adjacent to each other, piercing the opposing ends of the tissue with the needle, and closing the clamp by moving the first end of the body into proximity with the second end such that the needle extends into the opening.

Also disclosed is a device for applying surgical clips having a body with a first end terminating in a needle and a second end forming an opening thereat, the device preferably comprises an elongate first member having a first end and a second end with an aperture therebetween, an elongate second member having a first end and a second end with an aperture therebetween, a pin extending through the apertures to connect the first member to the second member such that the members are pivotable about the pin, and an anvil arranged at the second end of the second member. The anvil may be adapted to shape the needle of the clip when the second end of the first member is brought into proximity with the second end of the second member. The first ends of each member may form handles.

### BRIEF DESCRIPTION OF THE DRAWINGS

The subject matter regarded as the invention is particularly pointed out and distinctly claimed in the concluding portion of the specification. The invention, however, both as to organization and method of operation, together with features, objects, and advantages thereof will be or become apparent to one with skill in the art upon reference to the following detailed description when read with the accompanying drawings. It is intended that any additional organizations, methods of operation, features, objects or advantages ascertained by one skilled in the art be included within this description, be within the scope of the present invention, and be protected by the accompanying claims.

In regard to the drawings, FIG. 1 is perspective view of a suturing clip in accordance with one embodiment of the present invention;

FIG. 2 is a perspective view of a suturing clip in accordance with another embodiment of the present invention;

FIG. 3 is a perspective view of a suturing clip in accordance with yet another embodiment of the present invention;

FIG. 4 is a perspective view of a suturing clip in accordance with still another embodiment of the present invention;

FIG. 5 is a perspective view of a suturing clip in accordance with another embodiment of the present invention;

FIGS. 5A through 5C are side views of the suturing clip of FIG. 5 shown in various stages of closure, FIG. 5A being completely open and FIG. 5C being completely closed;

FIG. 6A is a perspective view of a suturing clip in accordance with another embodiment of the present invention in an open condition;

FIG. 6B is a perspective view of the suturing clip of FIG. 6A in a closed condition;

FIG. 6C depicts a perspective view of a surgical device for inserting a clip with a clip in accordance with one embodiment of the present intention;

FIG. 7A is a perspective view of still another embodiment of the suturing clip of the present invention shown in a partially closed condition; and,

FIG. 7B is a perspective view of the suturing clip of FIG. 7A shown in a fully open condition.

### DETAILED DESCRIPTION

In the following is described the preferred embodiments of the suturing clip of the present invention. In describing the embodiments illustrated in the drawings, specific terminology will be used for the sake of clarity. However, the invention is not intended to be limited to the specific terms so selected, and it is to be understood that each specific term includes all technical equivalents that operate in a similar manner to accomplish a similar purpose.

The vascular suturing clip of the present invention may be used to anastomose two pieces of tissue, such as one blood vessel to a second blood vessel. The suturing clips may also be utilized to anastomose vascular prosthetics to blood vessels or other organs. In addition, the suturing clips may be utilized to secure a single piece of tissue which has been split, such as in the case of an external wound to the skin tissue.

FIG. 1 depicts one embodiment of the vascular suturing clip of the present invention. As shown in FIG. 1, the vascular suturing clip 100 of this embodiment is formed from a single strand of wire 102, which is bent and formed into the suturing clip. The suturing clip 100 includes a first end 104, a second end 106 with a bite portion 108 therebetween. The extreme first end 104 of the suturing clip 100 forms a needle 110. The needle 110 increases in diameter from its point 112 to its base 113. At the base 113 is preferably a hemostatic footpad 114. It will be appreciated that the diameter of the hemostatic footpad 114 is typically greater than the diameter of the wire 102 used to form the suturing clip 100, as well as wider than the base 113 of the needle 110.

The suturing clip 100 extends from this first end 104 along an arcuate path to the bite portion 108. The bite portion 108 of the suturing clip of this embodiment is formed by wrapping the wire 102 twice around an axis 116 so as to create a spring 109. It will be appreciated that the strength of the spring 109 is a function of the tightness of the wrapped wire 102, the number of wraps and the diameter and material of the wire. Each of these elements may be modified so as to create a suturing clip 100 with the desired compression. For example, the bite portion 108 may include wire 102 wrapped four times, if increased spring compression is desired.

From the bite portion 108, the wire 102 forming the suturing clip 100 extends along an arcuate path towards the second end 106. As shown in FIG. 1, this arcuate path is a typically a mirror image of the arcuate path between the first end 104 and the bite portion 108 of the clip 100, such that the clip generally forms a circle.

The second end 106 of the clip 100 may form a needle receiving ring 118 having a cavity 120. Alternatively, the needle receiving ring 118 may be formed as a separate component and then bonded to the second end 106 of the clip 100. The needle receiving ring 118 of this embodiment is formed by wrapping the wire 102 of the clip 100 around a single circular bend, as shown in FIG. 1. Multiple circular bends may also be employed.

The needle receiving ring 118 and the cavity 120 are typically sized large enough that the point 112 of the needle 110 may enter the cavity 120 but the footpad 114 may not. In its default, or resting position, the suturing clip 100 of this embodiment is in a closed position, such that the footpad 114 is engaged with the needle receiving ring 118. Again, the clip 100 may be adapted such that a predetermined compression force is present between the footpad 114 and the needle receiving ring 118.

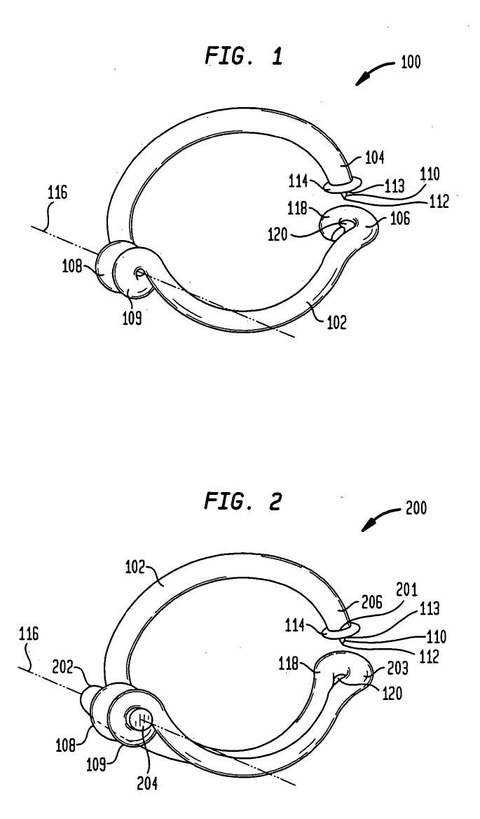

FIG. 2 depicts a suturing clip 200 in accordance with a second embodiment of the present invention. As with the first embodiment, the suturing clip 200 of the second embodiment is formed from a single wire 102. The clip 200 comprises a proximal end 201, a distal end 203, and a bite position 108 therebetween. A first end 204 of the wire 102 assists with forming the bite portion 108 of suturing clip 200. The second end 206 of the wire 102 forms a needle 110 at the proximal end 201 of the clip 200.

To form the suturing clip 200 of this embodiment, the first end 204 of the wire 102 is placed along an axis 116 around which the bite portion 108 of the suturing clip 200 is to be formed. The wire 102 is then bent along a first curve 202 towards the distal end 203 of the suturing clip 200. From the first curve 202, the wire 102 generally follows an arcuate path to the distal end 203. At the distal end 203, the wire 102 is looped around to form a needle receiving ring 118 and a cavity 120, similar to the like elements of the first embodiment. The wire 102 then extends back towards the bite portion 108 following an arcuate path matching the arcuate path followed from the first curve 202 to the distal end 203, where it is wrapped around the first end 204 of the wire 102 to form a spring 109. Again, the number of times the wire 102 is wrapped and the tightness of the wrapped wire will partially determine the strength of the spring. After being wrapped, the wire 102 then extends along an arcuate path towards the proximal end 201 of the suturing clip 200. As with the previous embodiment, the proximal end 201 includes a footpad 114 and a needle 110. The needle includes a point 112 which tapers from a bas 113 adjacent to the footpad.

It will be appreciated that the suturing clip 200 of the second embodiment is similar in operation to suturing clip 100 of the first embodiment. However, this embodiment of the suturing clip 200 is generally stronger than the first embodiment of the suturing clip 100 when like materials of like diameters are employed because of its geometry. As with the previous clip 100, this suturing clip 200 is closed in its resting, or default position, such that the footpad 114 is engaged with the needle receiving ring 118.

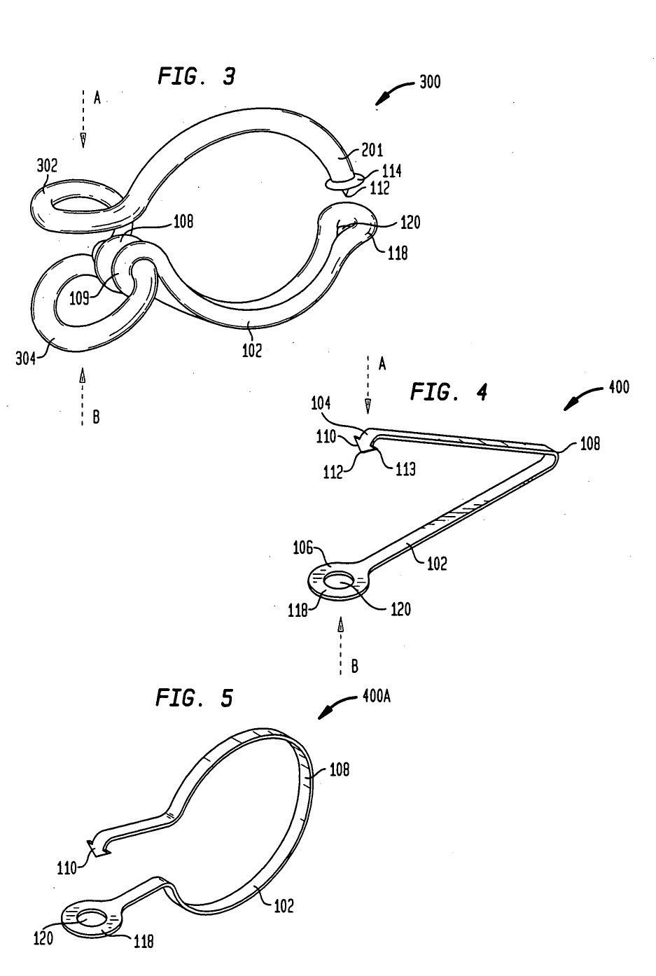

FIG. 3 discloses a suturing clip 300 in accordance with a third embodiment of the present invention. The suturing clip 300 is formed in a similar manner as the suturing clip 100 and the suturing clip 200. However, the suturing clip 300 includes a first leverage loop 302 and a second leverage loop 304. As is shown in FIG. 3, the leverage loops 302, 304 are located adjacent to the bite portion 108 of the suturing clip 300. Each loop 302, 304 is formed from the continuous wire 102. Generally, the first leverage loop is formed between the bite portion 108 and the proximal end 201 by adding an extra loop of wire 102. Meanwhile, the second leverage loop 304 is typically formed at the bite portion 108 in a similar manner, as shown in FIG. 3.

The leverage loops 302, 304 are utilized to open the suturing clip 300, as its resting or default position is closed. By simultaneously applying leverage to each loop generally in the directions indicated by arrows A and B of FIG. 3, pressure will be biased against the spring 109 formed at the bite portion 108 of the suturing clip 300. Sufficient pressure will open the suturing clip 300.

Pressure may be applied manually by the surgeon's fingers or by the use of a device specially adapted for this purpose. Such devices may be as simple as a standard pair of pliers or as intricate as a specialized device which may house a cartridge of suturing clips 300 with automatic reloading of subsequent clips 300 during installation. It will be appreciated that suturing clips 100 and 200 of FIGS. 1 and 2, as well as those yet to be presented, may also be installed manually or by a similar device having a cartridge of clips.

As previously mentioned, suturing clips 100, 200, 300 shown in FIGS. 1-3 are closed in their default or resting position. To install the clips 100, 200, 300, the clips must be spread apart such that the needle 110 is moved distant from the needle receiving ring 118. Two pieces of tissue (not shown) intended to be sutured may then be placed between the needle point 112 and the needle receiving ring 118. The clip 100, 200, 300 may then be permitted to return to its natural condition where the needle point 112 will pierce the tissue due to the biasing of the bite portion 108 of the clips 100, 200, 300. Such biasing will permit the needle 110 to penetrate both tissue portions until such point as the hemostatic footpad 114 prevents further penetration owing to its oversized relationship with cavity 120 and loop 118.

One of the features of suturing clips 100, 200, 300 is the interaction of the needle 110 with the footpad 114 and the needle receiving ring 118 with cavity 120. Namely, the needle 110 may pierce tissue to be sutured in a similar manner as many of the prior art references. However, this action alone may permit tearing of the tissue if any subsequent pulling occurs. Footpad 114 assists with preventing this from occurring. Footpad 114 applies pressure to the tissue between itself and needle receiving ring 118 across a portion of its surface area. Thus, the tissue is held in place by both the piercing of the needle 110 and the applied pressure of the footpad 114. In addition, the pressure applied by footpad 114 assists in maintaining hemostasis so the flow of blood is arrested at the pierced area.

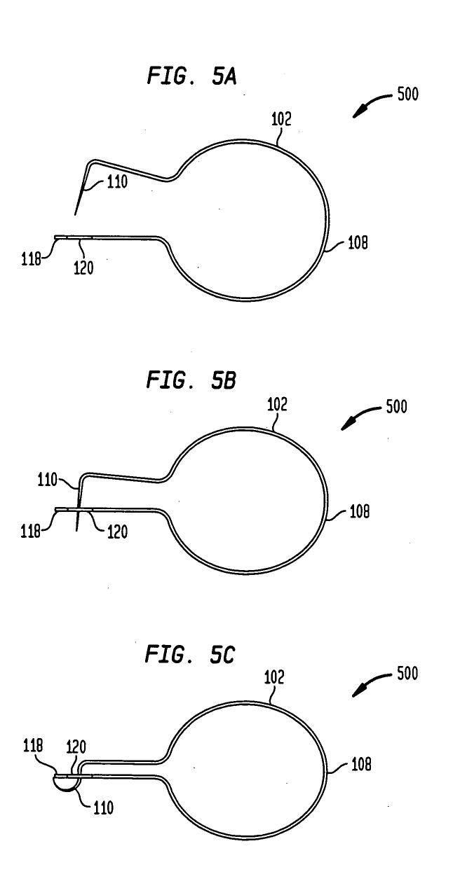

FIG. 4 depicts a suturing clip 400 in accordance with a fourth embodiment of the present invention. This suturing clip 400 is substantially similar to the previous suturing clips 100, 200, 300, except that its default or resting position is open and it includes a needle 110 which does not include a footpad 114 and is permitted to completely penetrate beyond the needle receiving ring 118 of the clip 400. In this regard, two pieces of tissue which are intended to be sutured may be placed between point 112 and looped needle receiving ring 118. The suturing clip 400 may be closed by applying pressure to the proximal end 104 and the distal end 106 in the directions indicated by arrows A and B, such that the point 112 pierces the tissue and extends beyond the cavity 120 formed by the needle receiving ring 118. The needle 110 may extend beyond the cavity 120 such that the base 113 completely passes through the needle receiving ring 118 and the cavity 120. To achieve such a result, the pressure must be sufficient to minimally deform the needle receiving ring 118 or the base 113 of the needle 110. Such deformation is available because of the nature of alloys used to form the clips. Also, the clips may have a non-continuous portion forming the needle receiving ring such as the clips shown in FIGS. 1-3, which are permitted to deform. Other clips, such as those shown in FIGS. 4 and 5, have a continuous needle receiving ring 118. These rings are formed by stamping the wire flat, and then punching out the cavity 120, or by other similar means. Of course, the wire 102 may also begin flat rather than cylindrical, such that the entire clip 400 will consist of flat wire.

Once closed in the such a manner, the base 113 of the needle 110 will be stuck beyond the cavity 120 preventing the suturing clip 400 from opening, despite the biasing of the bite portion 108 attempting to keep the clip 400 in an open condition. Thus, once the suturing clip 400 of this embodiment is engaged, it is difficult, if not impossible, to remove by non-destructive means. This suturing clip 400 may be applied manually by a surgeon or by the use of a specialized mechanical device which may include a cartridge of clips, as with the previous embodiments.

It will be appreciated that the bite portion 108 of clip 400 is bent with a relatively small radius. FIG. 5 depicts a similar clip 400A with a bite portion 108 bent into a larger radius. Either type of clip 400, 400A may be employed. Further, it will be noted that the bite portion 108 of clips 400, 400A is not wound to form a spring 109 as with the previous embodiments. Rather, the bite portion 108 comprises a simple arc through a given radius. This permits the clip 400, 400A to have an open default position, rather than closed as with the previous embodiments. Of course, the wire may also be bent into a spiral spring, if so desired.

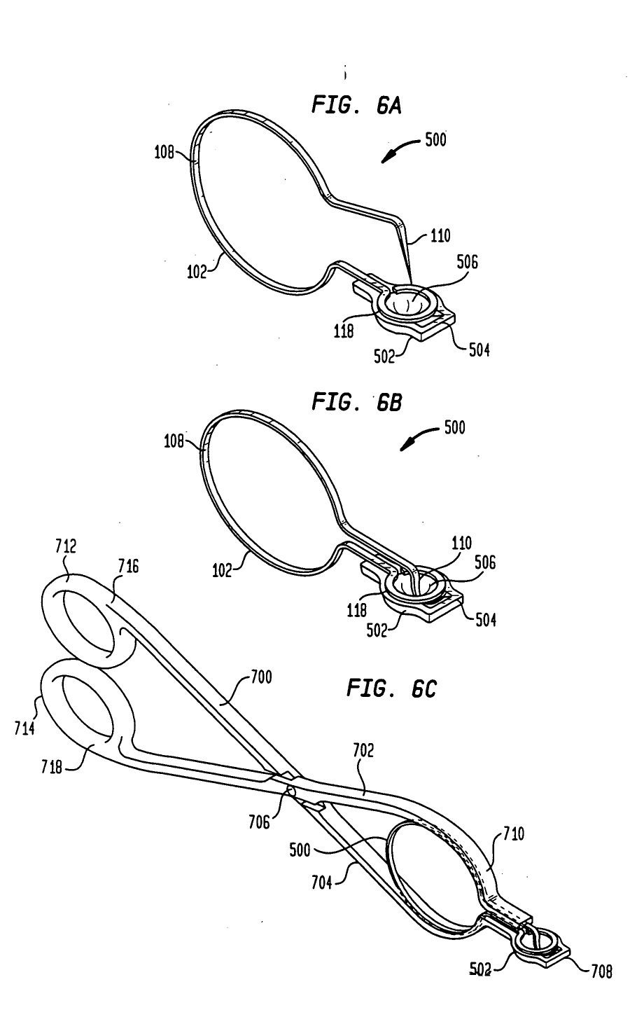

FIGS. 5A-5C depict a suturing clip 500 in accordance with a fifth embodiment of the present invention. Suturing clip 500 is substantially similar to the previous suturing clips 400, 400A. However, the needle 110 of the suturing clip of this embodiment does not include a base 113 extending beyond the outer diameter of the wire 102. Thus, when this clip is closed, there are no forces acting against the biasing force of the bite portion 108 to prevent opening of the clip other than a minor amount of friction between the needle 110 and the tissue to be clamped. Accordingly, the needle 110 must be bent below the level of the receiving ring 118 after the clip 500 is closed or the clip will open. FIG. 5B depicts a needle in an initial stage of being bent such that the suturing clip 500 will be prevented from opening. Such bending may be performed by a surgeon manually, such as with a special tool, for example, a pair of pliers. Preferably, a specialized device attached to the clip 500, such as an anvil bucket 502 (FIG. 6A) may be utilized. FIG. 5C depicts a suturing clip 500 having a needle 110 bent so as to prevent the clip from opening in accordance with this embodiment.

FIGS. 6A and 6B depict suturing clips 500 with an anvil bucket 502 attached. FIG. 6A depicts the suturing clip 500 in an open condition while FIG. 6B depicts the suturing clip in a closed condition. The anvil bucket 502 is typically attached to the clip 500 below the receiving ring 118 to bend the needle 110. The anvil bucket 502 typically comprises a relatively flat top surface 504 and a sunken shaped portion 506. As shown in FIGS. 6A and 6B, the shaped portion 506 may be curved from a shallow portion nearest the flat top surface 504 to its deepest portion near the center of the shaped portion such that the needle 110 may be gradually bent upon contact with the anvil bucket 502. As previously noted, the anvil bucket 502 may not be attached to the clip 500, but may in fact form a portion of a specialized applicator 700 for placing such clips 500, such as shown in FIG. 6C.

Such an applicator 700 may take the shape of a pair of pliers with two elongate members 702, 704 connected by a pin 706. One of the members may include the anvil bucket 502 at a distal end 708 while the other may include a mechanism 710 for grasping the clip 500. At the proximal ends 712, 714 of each member may be handles 716, 718, such as those formed in traditional scissors, so the surgeon may manipulate the applicator.

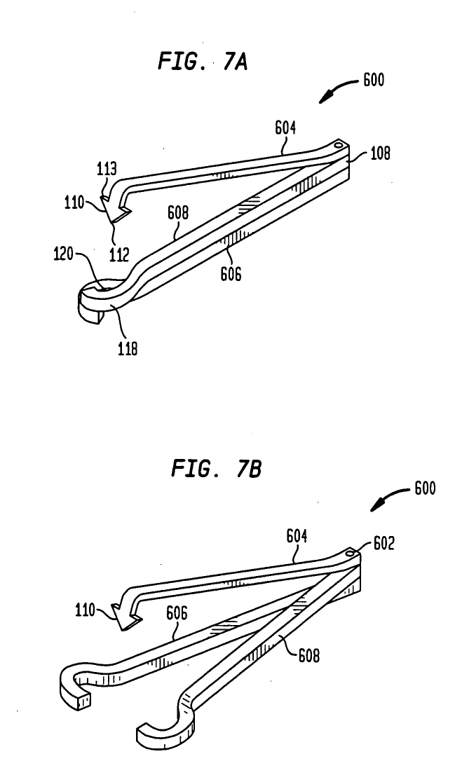

FIGS. 7A and 7B depict a suturing clip 600 in accordance with still another embodiment of the present invention. In this embodiment, the suturing clip 600 is formed from four pieces. Three of the pieces, a needle arm 604, a first extension 606 and a second extension 608 extend from the bite portion 108 of the suturing clip 600. Each of the pieces 604, 606, 608 is connected by, and is pivotable about, pin 602. Needle arm 604 extends from the pin 602 towards its terminus at needle 110. As with suturing clip 400, the needle 110 includes a point 112 and a base 113, which may generally form an arrow-shaped head. Each of the first extension 606 and the second extension 608 also extend from the pin 602 towards their respective terminus points which collectively form a needle receiving ring 118 with a cavity 120 when brought adjacent to each other. As shown in FIG. 7A, the first extension 606 and second extension 608 may partially overlap to form the needle receiving ring 118. However, they may also simply abut each other at their respective terminus points, if aligned on the same plane. Such abutment may still form a needle receiving ring 118.

First extension 606 and Second extension 608 may be pivoted about pin 602 such that they are in a closed position as shown in FIG. 7A or in an open position as shown in FIG. 7B. As more clearly shown in FIG. 7B, each of the first extension 606 and the second extension 608 form approximately one-half of the needle receiving ring 118.

The suturing clip 600 of this embodiment is installed in a similar manner as the suturing clip 400, 400A previously discussed. Namely, the first extension 606 and the second extension 608 may be spread apart such that the receiving ring 118 is not formed, as shown in FIG. 7B. The needle 110 may then be utilized to pierce the tissue to be sutured. The first extension 606 and second extension 608 may then be pivoted about pin 602 toward each other to form receiving ring 118 above the level of the base 113 of needle 110 so as to be positioned between the base 113 and the bite portion 108. As the natural biasing of the clip 600 is in the open position, it will be appreciated that such action will cause the needle 110 to remain below the level of the needle receiving ring 118 within cavity 120, placing the suturing clip 600 in a locked position.

Once the suturing clip 600 is in a locked position, the tissue will be secured. A feature of suturing clip 600 is that the suturing clip may easily be opened after being locked. To open the suturing clip 600, the first extension 606 and the second extension 608 may be spread apart as shown in FIG. 7A such that the needle arm 604 may be permitted to be withdrawn from penetrating the tissue. Thus, this suturing clip 600 has the benefits of being lockable as with the suturing clip 400, 400A, but also permits removal in a much simpler manner.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A suturing clip comprising a body having first and second ends, said first end terminating in a needle and said second end forming an opening thereat, said body adapted to manipulate said needle between a first and second position, said needle arranged spaced from said opening when in said first position and at least partially within said opening when in said second position.

2. The suturing clip of claim 1, wherein said body is formed from a single strand of wire.

3. The suturing clip of claim 1, wherein said first end further comprises a hemostatic footpad, said footpad having a dimension larger than said opening such that said footpad may not pass through said opening when said clip is in said closed position.

4. The suturing clip of claim 1, wherein said body further comprising a spring between said first end and said second end, said spring applying a biasing force to hold said body in either said first position or said second position.

5. The suturing clip of claim 1, wherein said needle is capable of being deformed when in said second position such that said needle will interfere with said opening to prevent said body from being placed in said first position.

6. The suturing clip of claim 1, wherein said body further comprises:
a bite portion between said first end and said second end;
a first needle arm extending from said bite portion to said first end;
a first extension member extending from said bite portion to said second end; and,
a second extension member extending from said bite portion to said second end.

7. The suturing clip of claim 6, wherein said first needle arm, said first extension member and said second extension member are connected to each other by a pin at said bite portion.

8. The suturing clip of claim 7, wherein said first extension member and said second extension member may be placed in proximity to each other at said second end to form said opening.

9. A suturing clip comprising:
a body having a first end, a second end and a bite portion therebetween;
said first end terminating in a needle; and,
said second end terminating with a needle receiving ring adapted to receive said needle;
wherein said bite portion applies a biasing force to hold said suturing clip in either one of a default open or a default closed condition, said needle being remote to said needle receiving ring in said open condition and in proximity with said needle receiving ring in said closed condition.

10. The suturing clip of claim 9, wherein said body is formed from a single strand of wire.

11. The suturing clip of claim 10, wherein said wire is either substantially cylindrical and substantially flat in cross-section.

12. The suturing clip of claim 9, wherein said first end further comprises a hemostatic footpad, said footpad having a dimension larger than said needle receiving ring such that said footpad may not pass therethrough when said clip is in said closed condition.

13. The suturing clip of claim 9, wherein said bite portion forms a spring.

14. The suturing clip of claim 9, wherein said needle is capable of being deformed when in said closed condition such that said needle will interfere with said needle receiving ring to keep said clip in said closed condition.

15. The suturing clip of claim 9, wherein said bite portion applies a biasing force holding said clip in a closed condition, said clip further comprising a first leveraging portion and a second leveraging portion arranged on opposite sides of said bite portion, said leveraging portions adapted to assist with opening of said clip.

16. The suturing clip of claim 9, wherein said bite portion applies a biasing force holding said clip in a closed condition, said clip further comprising a first leveraging portion and a second leveraging portion, said first leveraging portion arranged at said bite portion and said second leveraging portion arranged between said first leveraging portion and said needle.

17. The suturing clip of claim 9, wherein said bite portion applies a biasing force holding said clip in a closed condition, said clip further comprising a first leveraging portion and a second leveraging portion, said first leveraging portion arranged at said bite portion and said second leveraging portion arranged between said first leveraging portion and said needle receiving ring.

18. The suturing clip of claim 9, further comprising an anvil associated with said needle receiving ring, said anvil adapted to shape said needle when said clip is placed in said closed condition.

19. The suturing clip of claim 18, wherein said anvil comprises a relatively flat top surface and a shaped portion extending from a shallow portion nearest said flat top surface to a deep portion adjacent said shallow portion.

20. The suturing clip of claim 18, wherein said suturing clip is biased to be in the open condition.

21. The suturing clip of claim 9, wherein said body further comprises a needle arm extending from said bite portion to said first end, a first extension member extending from said bite portion to said second end, and, a second extension member extending from said bite portion to said second end.

22. The suturing clip of claim 21, wherein said first extension member, said second extension member, and said needle arm are connected to each other via a pin at said bite portion of said clip.

23. The suturing clip of claim 22, wherein said needle arm forms said needle at said first end of said clip.

24. The suturing clip of claim 21, wherein said first extension member and said second extension member form said needle receiving ring at said second end of said body.

25. The suturing clip of claim 22, wherein said first extension member and said second extension member are pivotable about said pin to form said needle receiving ring when said first extension member and said second extension member are brought adjacent to each other.

26. A suturing clip comprising a wire strand having a first end tapering into a needle, a second end configured to form a needle receiving ring to receive said needle, and a bite portion therebetween, said bite portion biasing said clip in either one of an open condition in which said needle is remote from said needle receiving ring and a closed condition in which said needle is at least partially within said needle receiving ring.

27. The suturing clip of claim 26, wherein said wire strand is substantially cylindrical.

28. The suturing clip of claim 27, wherein said wire strand forms a spring at said bite portion.

29. The suturing clip of claim 27, wherein said needle comprises a base and a point, said wire strand forming a footpad adjacent to said needle, said footpad having a dimension greater than the diameter of said wire.

30. The suturing clip of claim 29, wherein said footpad is positioned between said base of said needle and said bite position.

31. The suturing clip of claim 27, wherein said wire strand is shaped to form a spring circumscribing a portion of said wire strand at said bite portion of said clip.

32. The suturing clip of claim 26, wherein said wire strand is shaped to form a first leveraging portion and a second leveraging portion adapted to influence said bite portion to place said clip in said open condition.

33. The suturing clip of claim 32, wherein said first leveraging portion is formed from a single loop of said wire strand.

34. The suturing clip of claim 26, wherein said wire strand comprises a single length of wire between said needle and said bite portion.

35. The suturing clip of claim 26, wherein said wire strand comprises at least one length of wire between said bite portion and said needle receiving ring.

36. The suturing clip of claim 26, further comprising a anvil bucket associated with said needle receiving ring, said anvil bucket adapted to curve said needle to hold said clip in said closed condition.

37. A suturing clip comprising a first extension arm, a second extension arm and a needle arm, said first extension arm and said second extension arm being pivotable about a bite portion of said clip to form a needle receiving ring, said needle arm terminating with a needle opposite said bite portion, wherein said needle may penetrate said needle receiving ring to place said clip in a closed condition.

38. The suturing clip of claim 37, wherein said needle comprises a base tapering to a point, said base extending fully beyond said needle receiving ring in said closed condition.

39. A method of suturing opposing ends of severed tissue with a clamp having a body with a first end terminating in a needle and a second end forming an opening thereat, said method comprising:
placing the opposing ends of the tissue adjacent to each other;
piercing the opposing ends of the tissue with the needle;
closing the clamp by moving the first end of the body into proximity with the second end such that the needle extends into the opening.

40. The method of suturing of claim 39, wherein the body further comprises a footpad having a dimension larger than the opening, the footpad being adjacent to the needle at the first end, said method further comprising:
further closing the clamp such that said tissue is pinched between the footpad and the opening.

41. The method of claim 39, further comprising bending the needle.

42. The method of claim 39, wherein a specialized surgical device having an anvil is utilized, said method comprising bending the needle with the anvil of the specialized surgical device.

43. The method of claim 39, wherein the body further comprises a pair of leverage portions situated between the first end and the second end, said method further comprising applying pressure upon the leverage portions of the body to open the clip such that the needle is spaced from the opening.

44. The method of claim 39, wherein the body further comprises a bite portion between the first end and the second end and first and second extension members extending from the bite portion to the first second end, said method further comprising moving the first extension member and the second extension member of the body adjacent to each other to form the opening.

45. The method of claim 44, wherein said body further comprises a needle arm extending between the bite portion and the first end, said method further comprising bending the needle arm such that the needle passes beyond the opening.

46. A surgical device for applying suturing clips having a body with a first end terminating in a needle and a second end forming an opening thereat, said surgical device comprising:
an elongate first member having a first end and a second end with an aperture therebetween;
an elongate second member having a first end and a second end with an aperture therebetween;
a pin extending through said apertures to connect said first member to said second member such that said members are pivotable about said pin;
an anvil arranged at said second end of said second member, said anvil adapted to shape the needle of the clip when said second end of said first member is brought into proximity with said second end of said second member.

47. The surgical device of claim 46, wherein said second end of said first member is adapted to grasp the suturing clip.

48. The surgical device of claim 46, wherein said first end of said first member and said first end of said second member are formed into handles.
